(19) United States
(12) Patent Application Publication
(10) Pub. No.: US 2012/0289607 A1
(43) Pub. Date: Nov. 15, 2012

(54) ABSORBENT COMPOSITION AND METHODS THEREOF

(76) Inventors: Haishan Xiong, New Hope, PA (US); Mei Li, New Hope, PA (US); Thomas C. Bailey, San Mateo, CA (US); Shangbin Xiong, Tianjin (CN)

(21) Appl. No.: 13/497,913

(22) PCT Filed: Sep. 28, 2010
(86) PCT No.: PCT/US2010/050461

§ 371 Date: Mar. 23, 2012

Related U.S. Application Data
(60) Provisional application No. 61/277,620, filed on Sep. 28, 2009.

(51) Int. Cl. B01J 20/22; A01K 29/00; B01J 20/30; A61K 8/73; B01J 20/26

(52) U.S. Cl. 514/770; 502/62; 119/173

(57) ABSTRACT

An improved absorbent composition containing biodegradable natural ingredients is described. The composition absorbs liquid quickly, has a large water-absorbing capacity and an excellent water retention capability. The composition enables replacement of a significant amount of less or none biodegradable superabsorbent polymers (SAP) with plant and/or other natural ingredients, while achieving the liquid absorption properties similar to those of the widely used SAPs. The composition can be used as an absorbent in aqueous liquid absorption products, such as disposable personal hygiene products, rendering the products more environmentally friendly.

### CROSS-REFERENCE TO RELATED APPLICATIONS

This application is a Section 371 of International Application No. PCT/US2010/050461, filed Sep. 28, 2010, which was published in the English language on Mar. 31, 2011, under International Publication No. WO 2011/038374 A1, which claims the benefit of U.S. provisional patent application No. 61/277,620 filed Sep. 28, 2009, the disclosures of both applications are herein incorporated by reference.

### FIELD OF THE INVENTION

Embodiments of the present invention relate to an improved absorbent composition, methods of making and using the improved absorbent composition, and eco-friendly products comprising the improved absorbent composition. In particular, the improved absorbent composition comprises a natural ingredient having a natural hydrocolloid material treated with at least one porous aluminum silicate.

### BACKGROUND OF THE INVENTION

Disposable personal hygiene articles include a family of absorbent items, including baby diapers, training pants, feminine pads, adult incontinence pants and other related products. The basic design of these products includes an absorbent core that is comprised of absorbent materials encased in water-permeable sheets. Disposable baby diapers are one of the leading sources of solid waste in landfills. About 50 million diapers per day are disposed of in the United States alone. That translates into some 18-20 billion diapers going to landfills annually worldwide. Prior to being potty-trained, a child can produce an estimated ½ ton of disposable diaper waste each year. A typical child uses 5,000-6,000 diapers from birth until being potty-trained. Comprised primarily of petroleum-based materials such as polypropylene and polyethylene, disposable diapers will take an estimated 500-1,000 years to decompose. Yet, in spite of numerous press articles about the impact of disposable diapers on the environment, American consumers steadfastly prefer disposables over clothe as reflected in usage patterns, and they also generally show an unwillingness to sacrifice convenience for alternative approaches.

This consumer behavior, combined with today's mainstream product offerings which are generally not “eco-friendly”, create enormous pressure on the environment. According to the US Environment Protection Agency (EPA), 3.7 million tons of used diapers were thrown into trash in 2008, amounting to 1.5% of all municipal solid waste. Significant amounts of land are required to store waste from disposable diapers and related disposable absorbent hygiene products. Because most of the materials used in diapers and related hygiene products are petroleum-based, this waste will stay landfills for hundreds, if not thousands of years before chemically breaking down.

Virtually all of new designs for personal hygiene products use polyacrylate-derived super absorbent polymers (SAPs) in the product core. In a typical modern disposable diaper, the SAPs account for about 25%-30% of total diaper weight. SAPs absorb a large amount of water quickly, thus ensuring dryness of the product user's contact skin. Typically, these synthetic SAPs can absorb 100×-200× of water by weight within minutes and more importantly, hold the liquid under constant external pressure. Therefore the use of SAP as the principal fluid-absorbing material of hygiene products such as diapers has been increasing.

Some manufacturers are producing more “eco-friendly” diapers now. The focus of most of these disposable diapers is on the treatment (“chlorine free”) or sourcing (“sustainably harvested”) of the wood pulp. Just a few brands use starch-based sheets to replace the traditional petroleum-based non-woven materials which have dominated disposable diaper products since the 1960s. The overall performance of these products is typically comparable to that of regular disposable diapers. With no requirement of behavioral change by consumers, these new products hold the promise of good acceptance by the general public. The immediate benefits of these diapers include reduction in use of petroleum-based materials and better biodegradability. The hope is to reduce the landfill spaces required for disposable diapers in the long run.

While these new biodegradable diapers probably do not require nearly as long as diapers made from plastics to chemically breakdown (vs. 500-1,000 years), the cycle time will still be unreasonably long if disposed of in landfills. Most municipal trash landfills have anaerobic and sunlight-deprived ambient conditions which are not favorable to promotion of biodegradation. For example, even fifty-year-old newspapers have been found in landfills which are perfectly readable, despite being printed on biodegradable wood-based papers. Without proper compost facilities and efficient mechanisms to separate biodegradable from non-biodegradable components of these waste materials, which is the situation in most countries around the world including the United States, even these new eco-friendly disposable diapers will continue to pile up and claim more land without decomposing readily.

Thus, the ultimate goal in creating an eco-friendly diaper design is a biodegradable diaper with an ingredient that activates bacteria to begin breaking down the large carbohydrate molecules found in the component of these products, such as wood pulp used in the absorbent core. A key missing component of this design is a primer that promotes bacteria growth, resulting in the complete degradation of the end product after usage and disposal. Furthermore, to achieve biodegradability without sacrificing product performance, SAPs also need to be substituted.

The use of natural hydrocolloids for increasing the viscosity of aqueous liquids has been known for years. Such natural hydrocolloids include konjac or flours from plants of the family of the Araceae and in particular from species of the genus Amorphophallus. Konjac, which can be obtained from the tubers of the species Amorphophallus Konjak (U.S. Pat. No. 3,928,322) as well as from other plant sources, absorbs and retains large quantities of water relative to its dry weight and forms a viscous gel typically within half an hour after hydration. Due to its exceptional water retention capability, konjac is used in foods, cosmetics, and pharmaceutical products, to name just a few applications. Regular konjac, as commercially available, forms excessive amounts of lumps when mixed with water, even under agitation. This phenomenon makes it difficult for the material to maintain its absorbency under repeated assaults of liquid. More specifically, the fast hydration and dissolution of konjac results in “gel blocking” wherein the swollen gel prevents the liquid to penetrate the material fully. This significantly reduces the overall absorbing capacity of the material in a practical application. In addition, while konjac is highly desired for its ability to form hydrated gels, the gels formed from commercial konjac lack the stability that would be desired for the economical and efficient use of the compositions in their gel forms. Regular konjac gel loses a significant amount of its viscosity after a few hours at room temperature and therefore is not well suited for retaining aqueous fluids beyond a relatively short period of time, a critical performance consideration when applied in personal hygiene products. It would be desirable to provide compositions that would remain stable after swelling for an extended period for many industrial and household applications. There is a need for improvement to more effectively use konjac as an aqueous liquid absorbent.

Fibers from many beans are other examples of natural hydrocolloids, which bind water molecules through the hydrophilic groups on the surface of the polysaccharide chains. These fibers are readily available as the byproducts of industrial extraction for oil and protein. For example, soybean is routinely used for its oil and protein content. The leftover material has a high concentration of cellulose-like fibers with high water absorbing capabilities. The advantages of incorporating such fibers are two-fold: ready availability and cheap cost. However, among the major issues of using such fibers as an absorbent is that these have the tendency to release the water upon external pressure. The water retention capabilities of the bean fibers need to be improved.

U.S. Pat. No. 5,571,764 describes a material for absorbing water and aqueous fluids comprised of a natural product from the tuber of a plant from the family of Araceae and a synthetic polymer based on (co)polymerized hydrophilic monomers. A blend of natural starches with synthetic polymers is disclosed in this method presumably because these natural starches alone are inadequate substitutes to synthetic SAPs in speed of hydration and liquid retention. Despite the suggestion to add these natural ingredients to disposable absorbent products as described in this application, the physical properties of the hydrocolloids mentioned in the patent dictate that portions of the hydrocolloids dissolve in water. The glucomannan described was of regular commercial grade, which has been demonstrated to lack efficacy in liquid absorption, e.g., due to “gel blocking.” The simple linkage of the glucomannan particles and the synthetic SAP through polyglycol as described in this patent application may not provide the desired absorption and retention effects, thus significantly limiting the practical uses of the materials in their intended applications. Indeed, the described absorbent contained mostly SAP, only 1 to 20% by weight of the natural product.

In U.S. Pat. No. 6,580,014, an absorbent composition is described based on the use of an absorbent polysaccharide such as glucommannan that is specifically capable of reacting with a polyvalent metal ion. According to this patent, it is required that the polysaccharide, upon contact with a small amount of a high viscosity liquid, be dissolved or dissociated and diffused quickly in the high viscosity liquid to thereby fix the high viscosity liquid. Thus, it is preferred for the polysaccharide and the thickening article to contain no polyvalent metal ions. The described methodology is said to be applicable when the amount of the bodily liquid is relatively small, such as with loose feces or blood. It does not address the need for ordinary disposable diaper use where a large amount of urine needs to be absorbed within seconds.

U.S. Pat. No. 7,455,902 describes a composite polymer comprised of carboxyalkyl cellulose, galactomannan or glucomannan, and non-permanent intra-fiber metal crosslinkers. It describes a process whereas the carboxylalkyl cellulose is crosslinked to glucomannan through a multivalent metal ion, such as Al3+ compounds, Ti4+ compounds, Bi3+ compounds, B3+ compounds, and Zr4+ compounds. The composite then undergoes another crosslinking step with the same metal ion or a different one in a “water miscible” solvent to reduce sliminess and improve water retention. The absorbent performance of the composite polymer was said to match that of the SAP. The drawback however, is that the multi-step liquid-phase processing makes it expensive to produce. In addition, the use of a “non-permanent” multivalent ion crosslinker makes it questionable as to the utility of the composite polymer in personal hygiene products.

There is a compelling need for an improved, more biodegradable superabsorbent material that can substitute synthetic polymer gels in personal hygiene products as well as many other absorbent applications, which will help reduce the quantity of synthetic materials disposed of into the environment. Embodiments of the present invention relate to an improved, more biodegradable superabsorbent composition, as well as the methods and products related to the composition.

### BRIEF SUMMARY OF THE INVENTION

It is now discovered that treating a natural hydrocolloid material with a porous aluminum silicate significantly enhances the aqueous liquid absorption properties of the natural hydrocolloid material. An improved absorbent composition based on the natural hydrocolloid material treated with porous aluminum silicates quickly absorbs an aqueous liquid and retains the liquid even under external pressure. The improved composition has liquid absorption and retention properties comparable to conventional SAPs, e.g., rapid, simple hydration with stable viscosity maintenance and aqueous liquid retention over wide ranges of temperatures, externally applied pressures, and salinity concentrations. This composition naturally breaks down into simple sugar molecules within several months after hydration from normal use. The released simple sugar molecules provide a great carbon source for microbes, creating an environment that promotes the breakdown of other biodegradable materials in the disposed diaper and landfills. The absorbent composition improves the performance of many aqueous liquid absorption products, such as personal hygiene products, for the absorption of significant amounts of moisture, bodily fluids or other aqueous solutions, when the composition is used as an absorbent in the products.

In one general aspect, embodiments of the present invention relate to an absorbent composition comprising a natural ingredient, wherein the natural ingredient comprises at least one natural hydrocolloid material treated with at least one porous aluminum silicate and the weight ratio of the at least one natural hydrocolloid material relative to the at least one porous aluminum silicate is 1:0.15 to 1:0.7 in the natural ingredient.

In another general aspect, embodiments of the present invention relate to a method of preparing an absorbent composition. The method comprises incubating at least one natural hydrocolloid material with an aqueous solution comprising at least one porous aluminum silicate at room temperature for 2-30 minutes to obtain a natural ingredient, wherein the pH of the aqueous solution is 5 to 9, and the weight ratio of the at least one natural hydrocolloid material relative to the at least one porous aluminum silicate is 1:0.15 to 1:0.7 in the natural ingredient.

Another general aspect of the present invention relates to a product for absorbing an aqueous liquid. The product comprises a composition according to an embodiment of the present invention as an absorbent for the aqueous liquid.

In addition, the present invention also generally relates to an improved method of manufacturing a product for absorbing an aqueous liquid. In the method, the improvement comprises using an absorbent composition according to an embodiment of the present invention as an absorbent for the aqueous liquid.

In a preferred embodiment, the absorbent composition according to embodiments of the present invention comprises 25% (wt/wt) to 90% (wt/wt) the natural ingredient and 10%-75% (wt/wt) a synthetic ingredient having at least one superabsorbent polymer.

In another preferred embodiment of the present invention, a method according to embodiments of the present invention comprises:

mixing 25 to 90 parts by weight of the natural ingredient with 10 to 75 parts by weight of a synthetic ingredient at 20o C to 40o C for 5 to 60 minutes to obtain a mixture, wherein the synthetic ingredient comprises at least one superabsorbent polymer;

drying the mixture at a temperature below 90o C; and

grinding the dried product.

In yet another preferred embodiment of the present invention, the at least one natural hydrocolloid material used in the present invention comprises 20% (wt/wt) to 100% (wt/wt) konjac powders each having a particle size of 0.05 mm to 1.00 mm; the at least one porous aluminum silicate comprises at least one of sepiolite and bentonite; and the at least one synthetic ingredient comprises sodium polyacrylate.

Other aspects, features and advantages of the invention will be apparent from the following disclosure, including the detailed description of the invention and its preferred embodiments and the appended claims.

### DETAILED DESCRIPTION OF THE INVENTION

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein. It must be noted that as used herein and in the appended claims, the singular forms “a,” “an,” and “the” include plural reference unless the context clearly dictates otherwise.

A super absorbent based on natural materials is herein described. The composition comprises ingredients from natural sources, such as natural hydrocolloid materials treated with a porous aluminum silicate. The composition can further comprise synthetic ingredients, such as superabsorbent polymers (SAPs). Compositions according to embodiments of the present invention have liquid absorbing properties similar to that of pure SAP yet contain significant amount of natural ingredients that are readily biodegradable. For example, a composition according to an embodiment of the present invention can readily absorb 20×-30× of saline solution within seconds, which is comparable to the performance of many commercial grade synthetic SAPs. Aqueous liquid absorbing products, such as disposable personal hygiene products, comprising compositions according to embodiments of the present invention as the absorbent are also described.

As used herein, the term “natural hydrocolloid material” refers to any hydrocolloid of natural origin. A hydrocolloid, also referred to as “hydrophilic colloid,” is a colloid system wherein the colloid particles are dispersed in or spread throughout an aqueous liquid, thus allowing the colloid system to absorb a certain quantity of the aqueous liquid. A colloid system refers to a mixture in which two substances are interspersed between each other. Depending on the quantity of available aqueous liquid, a hydrocolloid can become different states, e.g., gel or sol (liquid), after absorbing the aqueous liquid. Hydrocolloids can be either irreversible (single-state) or reversible. For example, agar, a reversible hydrocolloid of seaweed extract, can exist in a gel and sol state. In general, hydrophilic colloid molecules have an affinity for water molecules and, when dispersed in water, become hydrated. Hydrated hydrocolloids swell and increase the viscosity of the system, thereby improving stability by reducing the interaction between particles and their tendency to settle.

Preferably, the natural hydrocolloid material absorbs water and forms a gel-like material, to thereby retain large quantity of aqueous liquid and potentially replace the petroleum-based SAP in various applications.

The natural hydrocolloid material can be prepared from any natural source, such as plants, animals or microorganisms. For example, glucomannan or konjac can be prepared from plants; agar and carrageenan can be extracted from seaweed; and gelatin can be produced by hydrolysis of proteins of bovine and fish origins.

In one embodiment of the present invention, the natural hydrocolloid material comprises a natural polysaccharide having polymeric carbohydrate structures formed of repeating units of saccharide(s) joined together by glycosidic bonds. These polysaccharide molecules contain a large amount of surface hydrophilic moieties that can attract water molecules.

Examples of the natural hydrocolloid material that can be used in the present invention, include, but are not limited to, konjac, soybean extract, mung bean extract, carrageenan gum, xanthan gum, alginate, guar gum, gellan gum, gum arabic, locust bean gum, and derivatives thereof.

As used herein, “konjac” or “konjac powders” refers to a natural hydrocolloid material that can be made from the tubers of the konjac plants, i.e., plants of the family of Araceae. Examples of suitable konjac plants include species of the genus Amorphophallus, such as A. rivieri, A. aldus, A. bulbifer, A. campanuiatus, A. giganteus, A. variabilis, A. titanum, A. konjak and A. virosus, more particularly Amorphophalus Konjac C. Koch. The principal functional soluble constituent of konjac is glucomannan, also called konjac mannan, a polysaccharide comprised mainly of D-glucose and D-mannose subunits. Glucomannan comprises mainly a straight-chain polymer, with a small amount of branching. The component sugars of glucomannan are β(1→4)-linked D-mannose and D-glucose in a ratio of about 3:2. Thus, the basic structure of konjac contains β-1,4-(d-mannose-d-glucose)n linear repeating units. Side chains branch out of the straight-chain polymer of D-mannose and D-glucose, e.g., via β-(1→6)-glucosyl linkages, β-(1,3) glycosidic bond, and/or α-(1→6)-linked galactose units. As used herein, the term “konjac” or “konjac powders” encompasses all forms of konjac or its derived glucomannan component that are available from all commercial sources or that can be prepared from the tubers of the konjac plants. For example, konjac can be prepared from the tubers of the plants in a manner known in the art, see, for example, Ullmanns Encyklopadie der technischen Chemie, 3, Ullmann's Encyclopedia of Industrial Chemistry, 3rd Edition, Volume 13, page 191 (1962).

In one commercial form, the Japanese have traditionally made “konnyaku” from the tuber of konjac plant. Commercial forms of konnyaku for food can be made from the konjac flour, which is obtained from the dried tuber of the plant. Konjac flour contains a variety of insoluble materials as well as a major amount of desirable water-soluble substances. Regular konjac flour is typically produced by slicing the tuber and removing the skin, drying the cut tuber, and then grinding to form the flour, which can be air classified to suitable particles sizes, e.g., from 60 to 80 US mesh, with removal of fines. Any form of konjac flour and its mannan-containing derivatives can be used in embodiments of the present invention.

In a preferred embodiment of the present invention, the at least one natural hydrocolloid material used in the composition comprises 20% (wt/wt) to 100% (wt/wt) konjac powders each having a particle size of about 0.05 mm to 1.00 mm, preferably about 0.10 mm to 0.90 mm, 0.20 mm to 0.80 mm, 0.30 mm to 0.75 mm, or 0.40 to 0.75 mm.

Fibers from soybean and mung bean are other examples of natural hydrocolloids that can be used in the present invention. These bean fibers contain a significant amount of complex polysaccharides, such as cellulose, hemicellulose, and pectin, which are known to have liquid absorbing capabilities. The soybean fiber is widely available commercially as a by-product after the extraction of oil and protein. The mung bean fiber is also commercially available. These bean fibers are the predominant components of bean extracts prepared from de-skinned beans and extracted off protein and fat contents. The bean fiber powders or granulates can be made by methods known in the art in view of the present disclosure. For example, the bean fiber powders can be made by de-skinning, boiling, grinding and drying of the beans, followed by extracting the protein and fat.

In a preferred embodiment of the present invention, the at least one natural hydrocolloid material used in the composition further comprises 0% (wt/wt) to 80% (wt/wt) dry soybean extract granulates and/or dry mung bean extract granulates each having a particle size of 0.05 mm to 1.00 mm, preferably about 0.10 mm to 0.90 mm, 0.20 mm to 0.80 mm, 0.30 mm to 0.75 mm, or 0.40 to 0.75 mm.

When bean extracts are used in compositions according to embodiment of the present invention, prior to being treated with the at least one porous aluminum silicate, the surface of the at least one natural hydrocolloid material is modified with a cross-linking agent, a cross-linking initiator and a surface modifier to improve the water absorption and retention capabilities of the natural hydrocolloid material.

According to an embodiment of the present invention, prior to the treatment with the at least one porous aluminum silicate, the at least one natural hydrocolloid material is modified with a method comprising the steps of:

mixing the at least one natural hydrocolloid material with a 12.5% to 33% (wt/wt) solution of the surface modifier dissolved in 60% (wt/vol) ethanol;

adding to the mixture the cross-linking agent and the cross-linking initiator to obtain a surface modification mixture comprising the cross-linking agent, the cross-linking initiator, and the surface modifier at the relative weights of 0.05%-0.5%, 0.05%-0.3% and 20%-100%, respectively, compared to the at least one natural hydrocolloid material; and

incubating the surface modification mixture at 30 oC to 50 oC for 1 to 3 hours in a sealed reactor or a reactor filled with N2 gas to obtain surface modified natural hydrocolloid material;

washing the surface modified natural hydrocolloid material with ethanol; and

drying the washed surface modified natural hydrocolloid material for subsequent use.

In preferred embodiments, the cross-linking agent is N,N′-methylene bisacrylamide, the cross-linking initiator is selected from the group consisting of potassium persulfate, ammonium persulfate and H2O2, and the surface modifier is monolauryl maleate.

With additional modification, the soybean and/or mung bean extract can absorb aqueous liquid similarly as that of the pure hydrocolloids, such as konjac. Because of their wide availability and lower costs in preparation, the modified soybean and/or mung bean extract provides a good substitute for pure hydrocolloids in an aqueous liquid absorbent.

Additional examples of natural hydrocolloids that can be used in the present invention are briefly described below.

Locust bean gum and guar gum have the backbone of d-mannose units linked via β-(1,4) glycosidic bonds. The side chains branch out via α-(1,5) bond with galactose. Locust bean gum has fewer d-galactose side chains than guar gum. The D-mannose to D-galactose ratio is about 3.9:1.

Sodium alginate is composed of β-(1,4)-d-mannuronic acid monomers and α-(1,4)-1-guluronic acid monomer. Depending on the types and origin, alginate can have three distinct chemical structures: only mannuronic acid (such as M-M-M-), only guluronic acid composition (-G-G-G-G-), or monomer alternate (-M-G-M-G-M-G-).

Xanthan gum is a microbial desiccation-resistant polymer prepared commercially by aerobic submerged fermentation from Xanthomonas campestris. It is an anionic polyelectrolyte with a β-(1,4)-D-glucopyranose glucan backbone with side chains of α-(3,1)-d-mannopyranose-β-(2,1)-D-glucuronic acid-β-(4,1)-D-mannopyranose on alternating residues. About 40% of the terminal mannose residues are 4,6-pyruvated and the inner mannose is mostly 6-acetylated.

Gellan gum is a water-soluble polysaccharide produced by Sphingomonas elodea. It has a repeating unit of [D-Glc(β1→4)D-GlcA(β1→4)D-Glc(β1→4)L-Rha(α1→3)]n, linked via α-1,3 bond.

Guar gum is extracted from the seed of the Cyamopsis tetragonoloba. Guar gum is a galactomannan, consisting of a β-(1,4)-D-mannopyranose backbone with branch chains from 6-positions linked to a-D-galactose (that is, 1,6-linked-α-D-galactopyranose). There are between 1.5-2 mannose residues for every galactose residue.

Carrageenan is derived from eucheuma seaweed. The basic structure of carrageenan is a linear polysaccharide made up of a repeating dissacharide sequence of α-D-galactopyranose linked 1,3 called the A residue and β-D-galactopyranose residues linked through positions 1,4 (B residues). There are at least three types of carrageenan with slightly different chemical properties, including cation-dependency.

In a preferred embodiment, the natural hydrocolloid material used in the present invention comprises a mixture of konjac and soy bean exact and/or mung bean extract, which can be obtained from normal commercial channels or prepared from the plants.

As used herein, the term “porous aluminum silicate” refers to a mixture of aluminum, silica, and oxygen that can be either a mineral, or combined with water to form a clay. The mixture can also include other elements. For example, a porous aluminum silicate can contain a mixture of SiO2, Al2O3, CaO, MgO, Na2O and small amount of Fe2O3. The chemical compositions and the relative amounts of each chemical compositions in mineral porous aluminum silicates can depend on the locations of the mine. Examples of porous aluminum silicates that can be used in the present invention include, but are not limited to, sepiolite, montmorillonite, attapulgite, bentonite and activated clay.

In a preferred embodiment, the porous aluminum silicate used in the present invention comprises at least one of sepiolite and bentonite.

As used herein, the term “super absorbent polymer” or “SAP” refers to a synthetic polymer that can absorb and retain large amounts of an aqueous liquid relative to their own mass. It absorbs aqueous liquid through hydrogen bonding with the water molecule. Thus, the ability of an SAP to absorb water depends on the ionic concentration of the aqueous liquid, e.g., the higher ionic concentration, the less absorption. Thus, SAP may absorb much more liquid from de-ionized water than from a saline solution. In addition, the total absorbency and swelling capacity of SAP are also controlled by the type and degree of cross-linking to the polymer. For example, low density cross-linked SAP generally has a higher absorbent capacity, swells to a larger degree, and has a softer and more cohesive gel formation, while high cross-link density polymers exhibit lower absorbent capacity and swell, are firmer, and can maintain particle shape even under modest pressure. SAPs are now commonly made from the polymerization of acrylic acid, a derivative from petroleum oil. SAPs are generally considered to be non-biodegradable.

Examples of SAP that can be used in the present invention include polyacrylic acid and sodium polyacrylate. Sodium polyacrylate, the most commonly used SAP, is made by blending acrylic acid with sodium hydroxide in the presence of an initiator to form a polyacrylic acid sodium salt. Other examples include, but are not limited to, SAPs made from materials such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile, etc.

The modern polyacrylate-based SAP is generally made in a 2-step polymerization/surface cross-linking process that is well documented, such as in U.S. Pat. No. 5,164,459, DE 4,020,780, and EPO 509,708. Such a method is sufficiently described in F. L. Buchholz et al., ed., “Modern Superabsorbent Polymer Technology,” Wiley-VCH, New York, N.Y., pages 97-108 (1998).

Preferably, the SAP used in the present invention has defined particle sizes of about 0.05 mm to 1.00 mm, preferably about 0.15 mm to 0.85 mm, 0.2 mm to 0.75 mm, or 0.3 mm to 0.75 mm.

As used herein, a “personal hygiene product” refers to any articles used in absorbing bodily fluids that have an absorbent core composed of liquid absorbents encased in a supportive, liquid permissive, non-absorbing shell. Examples of personal hygiene products include, but are not limited to disposable baby diapers, pull-ups, feminine sanitary pads, adult incontinence pants, and similar items.

The definition of “personal hygiene product” includes any design of a disposable product that utilizes a water/aqueous permeable top sheet, an absorbent core and a water/aqueous impermeable back sheet. The absorbent core is encased by the 2 sheets. The absorbent core can be made of any materials that absorb aqueous liquids. For example, the core can consist of wood pulps with or without synthetic polymer absorbents. In a personal hygiene product according to an embodiment of the present invention, the core comprises a composition according to an embodiment of the present invention as an absorbent.

In view of the problems with the art and the need for improvement, it is one objective of the present invention to provide an absorbent composition based on natural materials for absorbing aqueous liquid, which has the absorption and retention properties comparable to existing polyacrylate-based super absorbent, but with much improved biodegradability. The composition should satisfy the requirement of a disposable diaper and other personal hygiene products, e.g., to keep the contacting skin dry for an extended period of time, under pressure and at body temperature, without causing any significant adverse effect.

These and other objectives of the present invention are achieved by a novel absorbent composition comprising a natural ingredient, wherein the natural ingredient comprises at least one natural hydrocolloid material treated with at least one porous aluminum silicate and the weight ratio of the at least one natural hydrocolloid material relative to the at least one porous aluminum silicate is 1:0.15 to 1:0.7. For example, the weight ratio of the at least one natural hydrocolloid material relative to the at least one porous aluminum silicate in compositions according to embodiments of the present invention can be 1:0.15, 1:0.20, 1:0.25, 1:0.30, 1:0.35, 1:0.40, 1:0.45, 1:0.50, 1:0.55, 1:0.60, 1:0.65 or 1:0.70.

The present invention also relates to a method of preparing a composition according to an embodiment of the present invention by treating a natural hydrocolloid material with a porous aluminum silicate.

In one embodiment, the method comprises incubating at least one natural hydrocolloid material with an aqueous solution comprising at least one porous aluminum silicate at room temperature for 2-30 minutes to obtain a natural ingredient, wherein the pH of the aqueous solution is 5 to 9, and the weight ratio of the at least one natural hydrocolloid material relative to the at least one porous aluminum silicate in the natural ingredient is 1:0.15 to 1:0.7.

While not wishing to be bound by theory, it is believed that treatment with the porous aluminum silicate modifies the surface properties of the natural hydrocolloid material, which prevents or reduces the dissolution thus enhances retention of the natural hydrocolloid material upon interaction with water, overcomes the “gel blocking” problem known in the art, and allows ready access of water molecules to the internal hydrophilic groups on the natural hydrocolloid material, while maintaining the water absorption property of the natural hydrocolloid material. The treatment with the porous aluminum silicate also improves odor-absorbing capabilities of the fibers, a desirable trait for hygienic products.

In one embodiment, an absorbent composition according to an embodiment of the present invention further comprises a synthetic ingredient having at least one superabsorbent polymer to further improve the rate of hydration and the time of water retention. Preferably, the composition comprises 25% (wt/wt) to 90% (wt/wt) the natural ingredient and 10%-75% (wt/wt) synthetic ingredient.

The combined absorbent composition can be prepared by a method comprises:

mixing 25 to 90 parts by weight of the natural ingredient with 10 to 75 parts by weight of a synthetic ingredient at 20o C to 40o C for 5 to 60 minutes to obtain a mixture, wherein the synthetic ingredient comprises at least one superabsorbent polymer;

drying the mixture at a temperature below 90o C; and

grinding the dried product.

According to embodiments of the present invention, the combined absorbent composition comprises, by weight, 25%, 30%, 35%, 40%, 45%, 50%, 55%, 60%, 65%, 70%, 75%, 80%, 85%, or 90% a natural ingredient, and 75%, 70%, 65%, 60%, 55%, 50%, 45%, 40%, 35%, 30%, 25%, 20%, 15%, or 10% a synthetic ingredient.

While not wishing to be bound by theory, it is believed that treatment with the porous aluminum silicate activates surface groups on the natural hydrocolloid material, which in turn interacts with polyacrylate in the synthetic ingredient to form complex molecules that further improve water permeability, absorption and retention.

Another general aspect of the present invention relates to a product for absorbing an aqueous liquid, which comprises an absorbent composition according to an embodiment of the present invention as an absorbent for the aqueous liquid. The present invention also relates to a method of manufacturing a product for absorbing an aqueous liquid. As compared to the prior art method, the improvement in such method comprises using an absorbent composition according to an embodiment of the present invention as an absorbent for the aqueous liquid.

In view of the present disclosure, it is readily apparent to those skilled in the art that an absorbent composition according to an embodiment of the present invention can be used as to substitute SAP in many applications, including, but not limited to, the following:

storage, packaging, transportation (packaging material for water-sensitive articles, for example flower transportation, shock protection);

food sector (transportation of fish, fresh meat; absorption of water, blood in fresh fish/meat packs);

medicine (wound plasters, water-absorbent material for burn dressings or for other weeping wounds);

cosmetics (carrier material for pharmaceuticals and medicaments, rheumatic plasters, ultrasound gel, cooling gel, cosmetic thickeners, sunscreen);

thickeners for oil/water or water/oil emulsions;

textiles (gloves, sportswear, moisture regulation in textiles, shoe inserts);

chemical process industry applications (catalyst for organic reactions, immobilization of large functional molecules (enzymes), adhesive for agglomerations, heat storage media, filtration aids, hydrophilic component in polymer laminates, dispersants, liquefiers);

building construction, installation (powder injection molding, clay-based renders, vibration-inhibiting medium, assistants in relation to tunneling in water-rich ground, cable sheathing);

water treatment, waste treatment, water removal (de-icers, reusable sandbags); cleaning;

agriculture industry (irrigation, retention of meltwater and dew precipitates, composting additive, protection of forests against fungal and insect infestation, delayed release of active ingredients to plants);

fire protection (flying sparks)(covering houses or house walls with SAP gel, since water has a very high heat capacity, ignition can be prevented; spraying of SAP gel in the case of fires such as for example forest fires);

co-extrusion agent in thermoplastic polymers (hydrophilicization of multilayer films); and

production of films and thermoplastic moldings capable of absorbing water (for example agricultural films capable of storing rain and dew water; SAP-containing films for keeping fresh fruit and vegetables which can be packed in moist films; the SAP stores water released by the fruit and vegetables without forming condensation droplets and partly reemits the water to the fruit and vegetables, so that neither fouling nor wilting occurs; SAP-polystyrene co-extrudates for example for food packs such as meat, fish, poultry, fruit and vegetables); carrier substance in active-ingredient formulations (drugs, crop protection).

The following examples are presented to further illustrate and explain the invention but are in no way intended to limit the scope of the present invention. Unless otherwise indicated, all parts and percentages are by weight and based on the weight of the composition at the indicated stage of processing.

### EXAMPLE 1

### Surface Modification of Natural Fibers

Dissolved 10 g of monolauryl maleate in 40 ml 60% ethanol. Separately, mixed 35 g konjac and 35 g of soybean powder (after extracting oil and protein). Alternatively, one can use 35 g carrageenan instead of konjac. The powder particle size was between 20 mesh to 50 mesh. Added 0.1 g of ammonium persulfate and 0.02 g of N,N′-methylene bisacrylamide to the konjac/soybean mixture. Quickly and thoroughly mixed this solid with the monolauryl maleate solution mentioned above. Sealed the reactor to minimize oxidation. Alternatively, the reaction can be carried out in a nitrogen-filled reactor. Maintained the reactor at 30° C. for 3 hours. The content was washed in 60% ethanol and then retrieved by filtration. The solid was dried at 70° C. for 2-4 hours or until no further weight loss in 20 minutes. The drying step can also be done under vacuum to speed up the process. During the drying, some aggregation might occur. For further operations, the dried, large particles were ground down to diameters below 0.75 mm. For clarity purpose, the final product of this step is called BKS-1 hereunto.

### EXAMPLE 2

### Inorganic Modification of BKS-1

Thoroughly mixed 3 g of sepiolite in 45 ml of water until it forms a uniform, slush-like suspension. Adjusted the pH to neutral (5-9). The suspension was incubated at room temperature for 30 minutes. Added 10 g of the BKS-1 powder from Example 1 to this suspension. Thoroughly mixed then kept at 40° C. for 10-30 minutes. Then increased the temperature to 70° C. for about 3 hours to dry. Ground the dried solid to granules with diameters 0.71 mm +/−0.15 mm. For clarity purpose, the final product of this step is called BKS-2 hereunto.

In order to analyze the results of the modifications, absorption properties were measured using standard methodologies: Free Swell, Centrifuge Retention Capacity (CRC) and Absorbency Under Load (AUL). These measures are well known to persons in the trade of the art. Briefly, these methods are discussed below for illustration purpose.

Free Swell (also called tea bag test in the trade): the absorbent material is weighed then sealed in a nonwoven sealed bag and is soaked in an excessively large quantity of water (or other testing liquid) for 30 minutes. The fully hydrated absorbent is weighed again. The Free Swell is expressed in the ratio of weight of absorbed liquid to the dry weight of the absorbent:
Free Swell=(total weight of the bag−dry weight of the absorbent and bag)/dry weight of the absorbent

CRC: The test conducted the same way in Free Swell except that after the 30 minute absorption, the bag that contains the testing material is subjected to a 250 g centrifugation force for 3 minutes, as described in EDANA ABSORBENCY II 441.1-99. The weight of the bag is measured and the CRC is expressed as the ratio of weight of absorbed liquid after the centrifuge to the dry weight of the absorbent:
CRC=(total weight of the bag−dry weight of the absorbent and bag)/dry weight of the absorbent

AUL: 1 g of the absorbent material is distributed evenly at the bottom of a glass cylinder with an inner diameter of 60 mm. The cylinder sits on top of a thin layer of polyester gauze and then a piece of porous glass. The apparatus is placed inside a petri dish filled with water or saline such that the top of the liquid is flush with the top of the porous glass. A piston that is only slightly smaller than that of the cylinder (can move up and down freely inside the cylinder) is place on top of the absorbent material. In the testings hereinto, the total pressure on the absorbent materials is 0.4 pound per square inch (psi). Upon adding the liquid and the piston, the test is let stand for 60 minutes in room temperature. The apparatus is then dismantled, the wet weight of the absorbent materials measured. AUL is expressed the ratio of weight of absorbed liquid to the dry weight of the absorbent:
AUL=(wet weight of the absorbent−dry weight of the absorbent)/dry weight of the absorbent

All the absorbency characteristics hereon in are measured in 0.9% saline. For BKS-2,
Free Swell=30-50 g/g,
CRC=20-30 g/g
AUL=5-10 g/g (at 0.4 psi)

### EXAMPLE 3

In addition to the procedure described in Example 2, the modification was also carried out in simpler manner. Thoroughly mixed 3 g of bentonite and 10 g of BKS-1 powder from Example 1 at room temperature. Added 20 ml of water to the mixture and thoroughly mixed. Incubated the reaction at 35° C. for 30 minutes. Dried the mixture in a 064208 Duo-Vac Vacuum Oven (Lab-Line) at 70° C. for 25 minutes. Ground the dried solid to granules with diameters 0.71 mm +/−0.15 mm. The absorbent characteristics of this product were tested and in the same range of BKS-2 as described in Example 2.

EXAMPLE 4

### Modification of Hydrocolloid

If the starting plant materials are pure hydrocolloid, the surface modification step as described in Example 1 can be skipped. In one presentation, 10 g of konjac powder was with 45 ml suspension as described in Example 2. In yet another presentation, 10 g of xanthan was used to replace konjac. The mixing was done thoroughly and quickly. The reaction was carried out at room temperature for 5-30 minutes. The mixture was then dried at 70° C. for about 3 hours. Grind the dried solid to granules with diameters 0.71 mm +/−0.15 mm. For clarity purpose, the final product of this step is called MGK hereunto.

MGK is slightly better than BKS-2 with respect to the AUL value. For MGK, the absorbency characteristics are as follows:
Free Swell=25-40 g/g,
CRC=20-30 g/g
AUL=8-18 g/g (at 0.4 psi)

### EXAMPLE 5

### Making of jMGK

MGK as described above is more potent absorbent compared to BKS-2. Its absorbency properties are close to those of the SAPs. To further improve the performance, polyacrylate was introduced to the materials. Immediately post the reaction as described in Example 3, prior to the drying, add 10 g of sodium polyacrylate to the reaction mix of Example 3. It is important to mix the mixture thoroughly and quickly. After the mixing, the reaction was incubated at room temperature, without any disturbance, for 30-60 minute. The materials were dried at 75° C., then ground to particle size of 0.71 mm +/−0.15 mm. The particles smaller than the lower range can be taken back to the beginning of Example 4 for another round of reaction.

The absorbency performance of jMGK is very similar to that of commercial SAPs':
Free Swell=35-40 g/g,
CRC=25-30 g/g
AUL=22-25 g/g (at 0.4 psi)

### EXAMPLE 6

### Making of jBKS-2

Similar to Example 4, absorbency performance of the BKS-2 can be improved by sodium polyacrylate. Immediately post the reaction as described in Example 2, add 5 g of sodium polyacrylate to the reaction mix. It is important to mix the mixture thoroughly. After the mixing, the reaction in incubated at room temperature, without any disturbance, for 30-60 minute. Then the materials are dried at 75° C., preferably under vacumm, ground to particle size of 0.71 mm +/−0.15 mm. The particles which are smaller than the lower range can be taken back to the beginning of Example 4 for another round of reaction.

The absorbency performance of jBKS-2 is very similar to that of commercial SAPS':
Free Swell=33-40 g/g,
CRC=22-29 g/g
AUL=20-22 g/g (at 0.4 psi)

### EXAMPLE 7

### Examples of Applying the Composition to Personal Hygiene Products

Disposable diapers and feminine hygiene pads are constructed with an absorbent core containing material made from wood pulp, cotton, or other plant cellulose fibers. These fibers serve the primary purpose of rapidly wicking moisture away from the point of entry of liquid into the pad during consumer use; secondarily, these fibers help create a chamber where liquid remains trapped with some physical distance from the user's skin. Both roles serve the objective of keeping dry the skin of the product user. Super-absorbent particles are blended into this core to form the primary moisture storage area of the product.

There are a number of different ways to add super-absorbent mixture to a diaper or feminine hygiene pad during manufacturing. The two most common methods are provided by means of illustration and are in no way exhaustive.

In the first process, MGK are injected into the same feed stock stream that supplies the fibers of the diaper core. The absorbent pad is formed on a moving conveyer belt on top of which is placed a continuous web of moving material that, upon completion of the conversion process, will form the outermost layer to the finished product, i.e., the back sheet material which is furthest away from the consumer's skin during use. The super absorbent material and cellulose fibers are well mixed and dispersed in an upstream process before being placed on the web. Typically, positive pressure from nozzles is used to spray this blended mixture directly onto the web moving atop the conveyer belt, while negative pressure from a vacuum is also applied from below by means of a perforated conveyor belt to pull the mixture into position to form the core.

A second method involves applying the absorbent particles of the said composition on top of the surface of the core after it has been formed. Application of these particles is accomplished in a manner similar to the first process: positive pressure from nozzles, or a gravity feed, is used to inject these on top of the fibrous core as it moves along a conveyor belt. This “sandwich” construction tends to have a high concentration of super absorbent closer to the user's skin than in the first example. With this geometry, the super absorbent will tend to form a blocking layer upon hydration, inhibiting transportation of moisture into the core. Without the fibers and super absorbent well blended, as in the first example, it is more likely that the user experiences higher moisture levels on the skin.

There are other methods of adding super absorbent to a disposable diaper or related products during manufacture. However, in all cases the said composition is blended with other fibrous materials to achieve a balance between rapid wicking of moisture upon use, and then storage of the aqueous liquid in an absorbent gel material away from the user's skin.

The above description is for the purpose of teaching the person of ordinary skill in the art how to practice the invention and is not intended to detail all of those obvious modifications and variations which will become apparent to the skilled worker. It is intended, however, that all such obvious modifications and variations be included within the scope of the invention which is defined by the following claims. The claims are meant to cover the claimed components and steps in any sequence which is effective to meet the objectives there intended, unless the context specifically indicates the contrary.

I/we claim:

1. A composition comprising a natural ingredient, wherein the natural ingredient comprises at least one natural hydrocolloid material treated with at least one porous aluminum silicate and the weight ratio of the at least one natural hydrocolloid material relative to the at least one porous aluminum silicate is 1:0.15 to 1:0.7 in the natural ingredient.

2. The composition of claim 1 comprising 25% (wt/wt) to 90% (wt/wt) the natural ingredient and 10%-75% (wt/wt) synthetic ingredient having at least one superabsorbent polymer.

3. The composition of claim 2, wherein
the at least one natural hydrocolloid material comprises one or more selected from the group consisting of konjac, carrageenan gum, xanthan gum, alginate, guar gum, gellan gum, gum arabic, locust bean gum, soybean extract, mung bean extract, and derivatives thereof;
the at least one porous aluminum silicate comprises one or more selected from the group consisting of sepiolite, montmorillonite, attapulgite, bentonite and activated clay; and
the at least one superabsorbent polymer comprises one or more selected from the group consisting of sodium polyacrylate and polyacrylic acid.

4. The composition of claim 2, wherein
the at least one natural hydrocolloid material comprises 20% (wt/wt) to 100% (wt/wt) konjac powders each having a particle size of 0.05 mm to 1.00 mm;
the at least one porous aluminum silicate comprises at least one of sepiolite and bentonite; and
the at least one synthetic ingredient comprises sodium polyacrylate.

5. The composition of claim 4,
wherein the at least one natural hydrocolloid material further comprises 0% (wt/wt) to 80% (wt/wt) at least one of dry soybean extract granulates and dry mung bean extract granulates each having a particle size of 0.05 mm to 1.00 mm, the granulates are prepared from de-skinned soybean or mung bean, respectively, and are extracted off of protein and fat contents, and
wherein, prior to being treated with the at least one porous aluminum silicate, the surface of the at least one natural hydrocolloid material is modified with a cross-linking agent, a cross-linking initiator and a surface modifier.

6. The composition of claim 5, wherein the cross-linking agent is N,N′-methylene bisacrylamide, the cross-linking initiator is selected from the group consisting of potassium persulfate, ammonium persulfate and H2O2, and the surface modifier is monolauryl maleate, and wherein the relative weights of the cross-linking agent, the cross-linking initiator and the surface modifier compared to the natural hydrocolloid material are about 0.05% to 0.5%, 0.05% to 0.3% and 20% to 100%, respectively.

7. A method of preparing a composition, comprising incubating at least one natural hydrocolloid material with an aqueous solution comprising at least one porous aluminum silicate at room temperature for 2-30 minutes to obtain a natural ingredient, wherein the pH of the aqueous solution is 5 to 9, and the weight ratio of the at least one natural hydrocolloid material to the at least one porous aluminum silicate in the natural ingredient is 1:0.15 to 1:0.7.

8. The method of claim 7, further comprising:
mixing 25 to 90 parts by weight of the natural ingredient with 10 to 75 parts by weight of a synthetic ingredient at 20 oC to 40 oC for 5 to 60 minutes to obtain a mixture, wherein the synthetic ingredient comprises at least one superabsorbent polymer;
drying the mixture at a temperature of about 50 oC to about 90 oC; and
grounding the dried product.

9. The method of claim 8, wherein
the at least one natural hydrocolloid material comprises one or more selected from the group consisting of konjac, soybean extract, mung bean extract, carrageenan gum, xanthan gum, alginate, guar gum, gellan gum, gum arabic, locust bean gum, and derivatives thereof;
the porous aluminum silicate comprises one or more selected from the group consisting of sepiolite, montmorillonite, attapulgite, bentonite and activated clay; and
the synthetic ingredient comprises at least one of sodium polyacrylate and polyacrylic acid.

10. The method of claim 9, wherein
the at least one natural hydrocolloid material comprises 20% (wt/wt) to 100% (wt/wt) konjac powders each having a particle size of 0.05 mm to 1.00 mm;
the aqueous solution comprises 5% to 10% by weight of the at least one porous aluminum silicate selected from sepiolite or bentonite, the total weight of the aqueous solution is about 3 to 7 times of that of the natural hydrocolloid material; and
the synthetic ingredient comprises sodium polyacrylate.

11. The method of claim 10,
wherein the at least one natural hydrocolloid material further comprises 0% (wt/wt) to 80% (wt/wt) at least one of dry soybean extract granulates and dry mung bean extract granulates each having a particle size of 0.05 mm to 1.00 mm, the granulates are prepared from de-skinned soybean or mung bean, respectively, and are extracted off of protein and fat contents, and
wherein, prior to being treated with the at least one porous aluminum silicate, the surface of the at least one natural hydrocolloid material is modified with a cross-linking agent, a cross-linking initiator and a surface modifier using a method comprising:
mixing the at least one natural hydrocolloid material with a 12.5% to 33% (wt/wt) solution of the surface modifier dissolved in 60% (wt/vol) ethanol;
adding to the mixture the cross-linking agent and the cross-linking initiator to obtain a surface modification mixture comprising the cross-linking agent, the cross-linking initiator and the surface modifier at the relative weights of about 0.05% to 0.5%, 0.05% to 0.3% and 20% to 100% respectively, compared to the at least one natural hydrocolloid material; and
incubating the surface modification mixture at 30 oC to 50 oC for 1 to 3 hours in a sealed reactor or a reactor filled with N2 gas to obtain surface modified natural hydrocolloid material;
washing the surface modified natural hydrocolloid material with ethanol; and
drying the washed surface modified natural hydrocolloid material for subsequent use.

12. The method of claim 11, wherein the cross-linking agent is N,N′-methylene bisacrylamide, the cross-linking initiator is selected from the group consisting of potassium persulfate, ammonium persulfate and H2O2, and the surface modifier is monolauryl maleate.

13. The composition prepared by the method of claim 7.

14. A product for absorbing an aqueous liquid, comprising the composition of claim 1 as an absorbent for the aqueous liquid.

15. The product of claim 14 being selected from the group consisting of products for personal hygiene, food packaging, agricultural water retention, industrial and household spill control, healthcare, wire and cable water blocking, and oil field management.

16. The product of claim 15, being a baby diaper, a sanitary napkin, an incontinence article, a surgical absorbent, a bandage, a wound dressing, pet litter, or a food absorbent pad.

17. In a method of manufacturing a product for absorbing an aqueous liquid, the improvement comprises using the composition of claim 1 as an absorbent for the aqueous liquid in the product.

18. The method of claim 17, wherein the product is selected from the group consisting of products for personal hygiene, food packaging, agricultural water retention, industrial and household spill control, healthcare, wire and cable water blocking, and oil field management.

19. The method of claim 18, wherein the product is a baby diaper, a sanitary napkin, an incontinence article, a surgical absorbent, a bandage, a wound dressing, pet litter, or a food absorbent pad.
